(19) United States
(12) Patent Application Publication
(10) Pub. No.: US 2022/0339342 A1
(43) Pub. Date: Oct. 27, 2022

(54) SYSTEMS, METHODS AND HANDHELD PULSATILE ASPIRATION AND INJECTION DEVICES

(71) Applicant: Mark A. Anton, Newport Beach, CA (US)

(72) Inventor: Mark A. Anton, Newport Beach, CA (US)

(21) Appl. No.: 17/745,544

(22) Filed: May 16, 2022

Related U.S. Application Data
(63) Continuation of application No. 16/653,891, filed on Oct. 15, 2019.
Application No. 16/653,891 is a continuation of application No. 15/444,259, filed on Feb. 27, 2017, now abandoned.
(60) Provisional application No. 62/301,328, filed on Feb. 29, 2016.
(60) Provisional application No. 62/300,395, filed on Feb. 26, 2016.

(51) Int. Cl. A61M 1/00; A61M 5/20; A61M 5/32

(52) U.S. Cl. CPC A61M 1/815; A61M 1/75; A61M 1/84; A61M 5/20; A61M 5/329; A61M 2205/50; A61M 2202/08; A61M 2205/3331; A61M 2205/3584; A61M 2205/502; A61M 2205/52; A61M 2205/8206

(57) ABSTRACT

Systems, devices and methods for use in liposuction and infusion medical procedures including handheld devices, micro-cannulas, curved cannulas vacuum pressure changing features.

### CROSS REFERENCE TO RELATED APPLICATIONS

The present application is a continuation of U.S. patent application Ser. No. 16/653,891, filed Oct. 15, 2019, which is a continuation of U.S. patent application Ser. No. 15/444,259, filed Feb. 27, 2017, now abandoned, which claims priority pursuant to 35 U.S.C. § 119(e) to U.S. Provisional Patent Application No. 62/301,328, filed Feb. 29, 2016, and claims priority pursuant to 35 U.S.C. § 119(e) to U.S. Provisional Patent Application No. 62/300,395, filed Feb. 26, 2016, the disclosures of all of which are hereby incorporated by reference in their entireties.

### FIELD OF THE INVENTION

The subject matter described herein relates generally to systems, methods and related apparatuses for fat and other tissue aspiration and fat and other synthetic filler injections using cordless, handheld, pulsatile and non-pulsatile devices.

### BACKGROUND OF THE INVENTION

Liposuction is one of the most popular procedures in aesthetic surgery that is practiced around the world. Liposuction procedures generally include the removal of undesirable fat cells from the face and body, including thighs, breasts buttocks and other areas to improve contours by reshaping, slimming or achieving another desired effect. In some cases, this fat can then be inserted back into the patient's body in a different location in an effort to achieve a specific aesthetic effect.

A cannula is a hollow metal tube with a bullet shaped tip that may have various different types of hole configurations near the tip. During a liposuction procedure, a cannula is placed through the skin and into the fatty layer that the surgeon plans to reduce by removing fat cells. Once the cannula is placed under the skin, a vacuum pump generates negative pressure up to a maximum pressure level and maintains the pressure level at a relatively constant level. Most liposuction machines used today include a vacuum pump that generates a maximum of about 30 inHg of negative pressure, where 14.74 PSI=762 torr=1 atm. Tubing attaches the large suction machine from a typical location on the floor of the operating room to a sterile cannula on the operative field. Fat is aspirated or drawn by the negative pressure created by the vacuum through the holes in the cannula and into the body of the cannula before being deposited in a reservoir.

One example of a current hand-held micro-liposuction adipose harvester, processor and cell concentrator is disclosed in U.S. patent application Ser. No. 13/844,548 including devices and methods for aspirating adipose tissue with a portable device. This device includes a processing chamber, a cannula, a vacuum source, a filter or screen for separating connective tissue strands from adipose tissue, a digestion area and a product cell concentration chamber.

There many different liposuction machines in use currently, including ultrasonic assisted, laser assisted, power assisted and radiofrequency assisted liposuction machines, all of which use some form of vacuuming suction. However, there are currently no liposuction machines that utilize pulsatile vacuum. It would be desirable to have pulsating components, features and liposuction devices in order to achieve beneficial effects. For example, it would be desirable to agitate and loosen the fat cells targeted for removal to increase the speed, efficiency and effectiveness of liposuction procedures, in addition to aspiration of other tissues for biopsy.

Further, there are currently no handheld motorized liposuction devices that are able to remove or harvest fat while remaining sterile, that can then rapidly or immediately re-inject this harvested fat into other areas of the face or body. At present, modification or improvement of facial contours and wrinkles is achieved using syringes containing synthetic fillers or autologous fat in an office setting. However, there no similarly simple handheld device exists that is also easily remove undesirable fatty bulges, for example in the neck, jowls and lateral to the nasolabial folds.

An additional issue existing in the reinjection art occurs as a result of suctioned fat being frequently accompanied by thicker clumps of fascia and fat. This can cause an obstruction of an operative cannula that can hinder and reduce the effectiveness of reinjection procedures. In these situations, manual thumb pressure of the syringe plunger by a user can result in over-pressurization until a sudden release of an undesirable fat bolus occurs. This can cause numerous aesthetic problems, especially when the reinjection site is meant to fill or affect a small, localized area. A motorized method of reinjection can provide a calibrated, smooth and reliable injection or reinjection for biological and synthetic fillers of different consistencies, especially relatively thick or dense ones.

All suction cannulas currently used are straight and no suction cannulas are produced that include any curvature. One reason suction cannulas are straight is because some of the technology in use that is incorporated into current suction cannulas requires the cannulas to be straight. However, as described herein, there are numerous benefits that can be provided by using cannulas that are curved to follow the curved planes of the body

It is therefore desirable to have a pulsating vacuum device or pulsating components associated with a vacuum device in order to achieve this agitation and loosening of the fat cells while remaining sterile. These devices and components also have applicability in other procedures and can aid in the agitation and loosening of other cells and tissue, such as fine needle aspiration (FNA) biopsy and micro-tissue harvesting.

### SUMMARY OF THE INVENTION

Disclosed are systems, devices and methods for use in liposuction and other medical procedures. These include devices and components that include fixed and variable pulsatile vacuums. For variable pulsatile vacuum devices and components, frequency and amplitude of pulse oscillations can be adjusted before or during a procedure to achieve specific effects. As such, various pulsating component mechanisms can be applied to different liposuction techniques and volumes of cells and tissues to be removed.

Additionally, disclosed are systems, methods and handheld micro-cannular suction devices that can be cordless, battery-operated and motorized and that can be used to remove small volumes of fat cells. One example of a use for such devices is in facial and neck-related liposuction procedures. In some embodiments, patients can even remain awake during procedures. In some embodiments, these handheld, mechanized devices can have operability for both suction and injection procedures that allow a surgeon to easily harvest fat and then quickly reinject it into desired areas that are selected to benefit from augmentation

Also disclosed herein are curved cannulas and methods of use for liposuction procedures. These cannulas provide numerous benefits, including the ability to stay in one or more optimal tissue planes of the endlessly curved body. As such, curved cannulas can assist a surgeon in maintaining the tip of the cannula in the correct planes between the skin and underlying structures during medical procedures, thereby greatly minimizes the risk of contour irregularity. Curved cannulas also allow the surgeon to greatly minimize the number of incisions needed to sculpt the entire body as the curved cannula can spiral around the sides of the body from the anterior or posterior aspects. Since curved cannulas allow surgeons to follow bodily curves and spirals, they can provide reduced time for healing, more effective healing and increased ease and effectiveness during procedures.

### BRIEF DESCRIPTION OF THE DRAWING(S)

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely. Illustrated in the accompanying drawing(s) is at least one of the best mode embodiments of the present invention.

FIG. 1 shows an example embodiment diagram of a liposuction system.

FIG. 2 shows an example embodiment internal component diagram of a peristaltic pulsator.

FIG. 3 shows an example embodiment of a liposuction system.

FIG. 4 shows an example embodiment diagram of a piston head, piston plunger arm and eccentric gear.

FIG. 5 shows an example embodiment diagram of a handheld micro-cannular suction device.

FIG. 6 shows an example embodiment of a handheld micro-cannular suction device coupled with a syringe.

FIG. 7 shows an example embodiment of a volume aspirated versus mmHg graph.

FIG. 8 shows an example embodiment diagram of a plurality of curved cannulas with varying lengths and curvatures.

FIG. 9 shows an example embodiment of a human body frontal diagram 900 and human body backside diagram showing various curved liposuction pathways for curved cannula.

FIG. 10 shows an example embodiment diagram of two cannulas.

FIG. 11 shows an example embodiment of a controller architecture diagram for use with a handheld micro-cannular suction device.

FIG. 12 shows an example embodiment of a component diagram for coupling a syringe plunger with a screw drive.

FIG. 13 shows an example embodiment diagram of a plunger and rubber tip.

FIG. 14 shows an example embodiment diagram of a rubber tip and screw.

FIG. 15 shows an example embodiment diagram of a handheld micro-cannular suction device.

FIG. 16 shows an example embodiment of a drive motor.

### DETAILED DESCRIPTION

Before the present subject matter is described in detail, it is to be understood that this disclosure is not limited to the particular embodiments described, as such may vary. It should also be understood that the terminology used herein is for the purpose of describing particular embodiments only, and is not intended to be limiting, since the scope of the present disclosure will be limited only by the appended claims.

As described herein, a pulsating vacuum with adjustable or otherwise variable pulse frequencies and amplitudes can help agitate fat cells and ease in their removal. This technology can be applied to many of current and later developed liposuction techniques and procedures. The components, devices and features described herein can also be applied to fine needle aspiration (FNA) biopsy procedures in order to improve specimen loads.

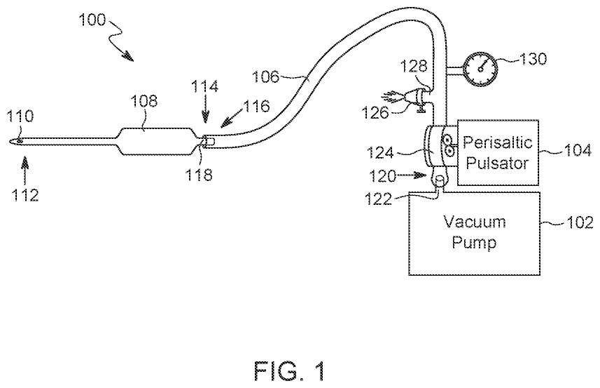

FIG. 1 shows an example embodiment diagram of a liposuction system 100. As shown in the example embodiment, liposuction system 100 can generally include a vacuum pump 102, a pulsator 104, tubing 106 and a cannula 108.

As shown in the example embodiment, cannula 108 can include one or more holes 110 at a cannula distal end 112 and can be removably or permanently coupled with a distal end 116 of tubing 106 at cannula proximal end 114. One or more components 118 can be used to assist in coupling tubing 106 and a cannula 108, such as screwing mechanisms, clamps, latches or others. In various embodiments, components can be sized such that cannula proximal end 114 can be inserted into distal tubing end 116, distal tubing end 116 can be inserted into cannula proximal end 114 or they can be coupled without inserting one into the other. Cannula 108 can have a larger section.

Tubing 106 can be one or more pliable tube that is hollow, has an open distal end 116 and an open proximal end 120. As shown in the example embodiment, open proximal end 120 can be removably coupled over a nozzle 122 of vacuum pump 102. In other embodiments, open proximal end may be inserted into a hole in vacuum pump 102. Material for tubing 106 can be a single uniform material or can be multiple materials. As such, tubing 106 can have different qualities and features in different locations. For example, more rigid sections can be interspersed with more pliable sections. Tubing 106 can have a circular cross-section in some embodiments that may be uniform or may be different at different locations. One example is a section that has accordion-like features.

As shown in the example embodiment, vacuum pump 102 can be coupled with tubing 106 and tubing 106 coupled with cannula 108 can create a sealed environment in which air, fluids and bodily materials can be drawn through hole 110 of cannula 108, into tubing 106 and into vacuum pump 102. In the example embodiment, a Luer-lok type tip can be used to securely couple tubing 106 to vacuum pump 102. In some embodiments, material for tubing 106 with less elastic wall properties or walls that have more rigid properties can improve the propagation of pressure fluctuations from a vacuum pump device 102 to the distal tip 112 of cannula 108.

In various embodiments, vacuum pump 102 can be located on a floor, table, stool, box or in another location. When connected to electricity and turned on, a motor in vacuum pump 102 creates a negative pressure at a directed source, here nozzle 122. In some embodiments, vacuum pumps can be manually controlled. In some embodiments, they can be semi-automatically or automatically controlled using computer processor and instructions stored in memory. User interface displays may allow users to change settings and modify vacuum pump conditions. Vacuum pumps are generally known to create movement, flux or changes in location in a fluid.

Regarding pulsations, pulses or other fluctuations in suction pressure generated by vacuum pump 102, these can be achieved using a variety of mechanisms, such as peristaltic pumps, piston pumps, motorized reciprocating screw drives or others. As shown in FIG. 1, constant negative pressure created in tubing 106 by liposuction vacuum pump 102 can be manipulated or otherwise fluctuated by inserting a portion of tubing in a channel 124 within a portion of or otherwise associated with pulsator 104. The example embodiment shows pulsator 104 as a peristaltic pulsator but it can be a different pulsator in different embodiments.

Generated fluctuations in pressure within tubing 106 can be accentuated in various embodiments by including a relief valve 126 that is coupled with an opening 128 in a side of tube 106. Relief valve 126 can have a reduced diameter compared with opening 128 and in the example embodiment it is shown as in a location on tubing 106 that is distal to the location of peristaltic pulsator 104, such that it is positioned between pulsator 104 and the patient. Timing for affecting an opening or closing of air valve 126 such that it is synchronized, desynchronized or offset from operations performed by peristaltic pump 104 can further accentuate the pressure fluctuations.

Also shown in FIG. 1 is a pressure gauge 130 that is coupled with tubing 106. Pressure gauge 130 can include a digital or analog mechanism for displaying an internal pressure of tubing 106 such that a surgeon, nurse or other party can monitor operating conditions.

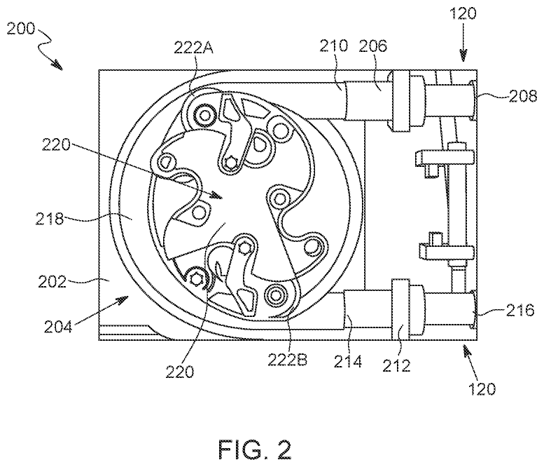

FIG. 2 shows an example embodiment internal component diagram of a peristaltic pulsator 200. As shown in the example embodiment, a pulsator body 202 can be constructed of one or more materials including plastic, metal or others as appropriate. A channel 204 in body 202 can be defined by one or more walls and can have various cross sections including circular, semicircular, square, rectangular, triangular or others. In the example embodiment, channel 204 has an overall semicircular shape.

A first tube interface 206 includes a hollow interior tunnel defined by at least one wall having a length and ending in a fluid inlet 208 and fluid outlet 210. Similarly, a second tube interface 212 includes a hollow interior tunnel defined by at least one wall having a length and ending in a fluid inlet 214 that is opposite a fluid outlet 216.

As shown in the example embodiment, pulsator internal tube 218 can be pliable and can be coupled at each end with fluid inlet 214 and fluid outlet 210 respectively. Internal tube 218 can be placed in channel 204 and partially wrapped around rotor 220.

Rotor 220 can be a mechanical or electro-mechanical rotating mechanism that is driven by a motor (not shown) and rotates about a central axis 220. As shown one or more arms or projections 222a, 222b can extend away out from central axis 220 and extend at least partially into channel 204. In operation, projections 222a, 222b contact internal hose 218 and can pinch or otherwise influence pressure within internal tube 218.

When a vacuum is operating, for example according to the principles and techniques described with respect to FIG. 1, fluid is drawn by a vacuum through fluid inlet 208, fluid outlet 210, tube 218, fluid inlet 214 and fluid outlet 216. Rotator 220 can then influence the flow regularly or irregularly based on the speed it is operated at.

It should be understood that tubing can be coupled with fluid outlet 216 and fluid inlet 208. Additionally, tubing 218 can be part of a larger single tube that is looped into first tube interface 206 and second tube interface 212.

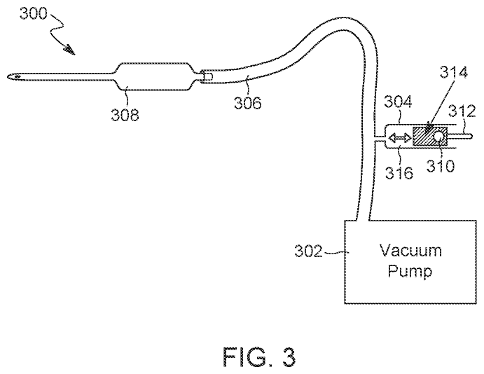

FIG. 3 shows an example embodiment of a liposuction system 300. As shown in the example embodiment, liposuction system 300 can generally include a vacuum pump 302, a pulsator 304, tubing 306 and a cannula 308. The setup and interfaces can be similar to those described with respect to FIG. 1, with the exception of pulsator 304. As mentioned herein, a pulsator 304 can be a mechanism that has a piston design.

As shown in the example embodiment, pulsator 304 can be an air sealed piston having a head 310 and plunger 312 within a chamber 314. Chamber 314 can be cylindrical and can be located within an airtight cylinder body 316 coupled in a T-connector fashion with tubing 306. The piston plunger 312 can be connected in an eccentric fashion to a toothed gear that can be round. The gear can be connected to a mechanical or electromechanical motor that, when powered drives the gear to rotate. When the motor rotates the gear, the piston head 310 and plunger 312 forward and backward and creates fluctuations and other pulses in tubing 306.

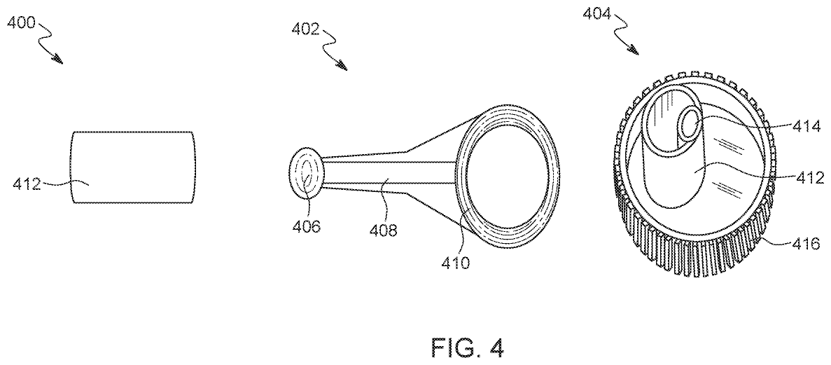

FIG. 4 shows an example embodiment diagram of a piston head 400, piston plunger arm 402 and eccentric gear 404. As shown in the example embodiment, a piston plunger arm 402 can include a distal end 406, a body 408 and proximal receiver end 410. Piston head 412 can be generally cylindrical and sized to be airtight within a piston chamber. Piston head 412 can be coupled with distal end 406 of plunger arm 402. Proximal receiver end 410 of piston arm 402 can be rotatably coupled with an exterior surface of driving cylinder 412 eccentric gear 404. A central hole 414 can maintain a standard position when rotatably coupled with a pin or other appropriate component. Ridges 416 can be engaged with ridges of another component or motor in order to cause eccentric gear 404 to rotate.

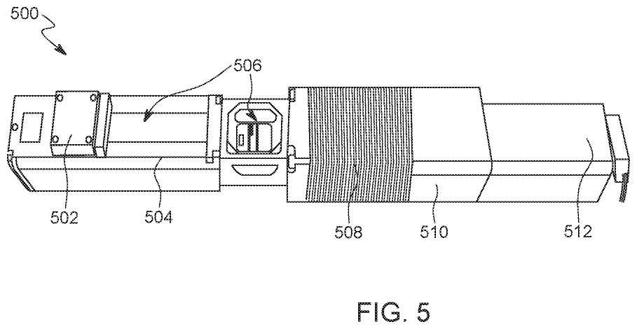

FIG. 5 shows an example embodiment diagram of a handheld micro-cannular suction device 500. As shown in the example embodiment, device 500 can generally include a platform body 504, movable platform 502, screw drive 506, motor 508, smart controller 510 and power source 512. In some embodiments, device 500 can also be used for infusion. In some embodiments device 500 can be used exclusively for infusion.

As shown in the example embodiment, moving platform 502 can be slidably coupled with platform body 504 such that moving platform can move laterally across at least a portion of a face of platform body 504 through an opening or channel in the face. In the example embodiment moving platform is removably or permanently coupled with a feature (not shown) that is operable to be moved in one direction and a second direction using screw drive 506 when powered by motor 508. This feature can be a threaded cylindrical shape in some embodiments, with threads corresponding to those of screw drive 506. As screw drive is cylindrical and coupled with a rotating component of motor 508, it can turn or rotate on a central axis in both directions, depending on the direction motor 508 causes it to turn or rotate. Motor 508 can be electrically and communicatively coupled with smart controller 510 that includes instructions, stored in non-transitory memory, that operate motor 508 according to a program. Smart controller 510 can also include at least one processor that executes the instructions. Motor 508 and smart controller 510 can be electrically coupled with and powered by a removable or rechargeable battery 512.

Additional features can also be provided in various embodiments, including a housing; power and operation buttons; potentiometers and other sensors; user interface displays showing tension level readouts, pressure level readouts, timers and other pertinent information; LED or other indicator lighting and others as would be appropriate in order to assist the user or operator during a procedure.

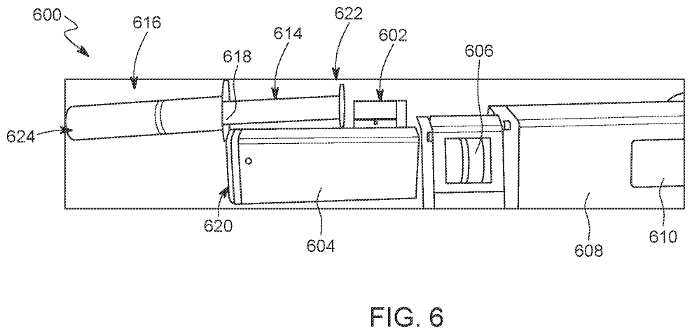

FIG. 6 shows an example embodiment of a handheld micro-cannular suction device 600 coupled with a syringe 616. As shown in the example embodiment, device 600 can generally include a platform body 604, movable platform 602, screw drive 606, motor 608, smart controller 610 and power source (not shown) with couplings and operation similar to the description of FIG. 5. Although not shown, a housing for devices 500 and 600 can be provided that protects the interior components and allows for operation as described herein without impediment.

In some embodiments, motor 608 can be a step motor that is coupled with screw drive 606. In some embodiments motor 608 can be a brushless motor. This can power movement of moving platform 602, which can be removably coupled to a plunger 614 of syringe 616. As shown, a lip 618 (or other appropriate location) of syringe 616 can be removably coupled with a distal end 620 of device 600 in order to maintain it in a stationary position with respect to plunger 614 in operation, such that plunger 614 can expel, dispense or otherwise push out the contents within syringe 616. Similarly, in reverse operation, plunger 614 of syringe 616 can draw in external fluid or matter into syringe 616 when motor 608 is run in a reverse direction that causes moving platform 602 to move along screw drive 606 proximally with respect to body 604. In some embodiments, the syringe is about 1-20 cc, while in some embodiments it is 10-12 cc or others.

As described previously, syringe plunger 614 can be coupled with moving platform 602 and controlled by the motor-coupled screw drive 606. In some embodiments, step motors or brushless motors are coupled with screw drives that are in turn coupled to a plunger of a syringe of about 1 cc to about 30 cc or more. In some embodiments screw drive 606 can be coupled with a center of the plunger 614 itself. This can be achieved by coupling the screw drive 606 to the plunger 614 or by having a sterile adaptor (not shown) in between plunger 614 and screw drive 606. This can also be accomplished by coupling screw drive 606 to a handle at the proximal end 622 of plunger 614. Syringe 616 and its various components can be rubber or other appropriate materials in various embodiments and can be disposable and sterile.

In various embodiments, a disposable sterile needle or cannula can be coupled with the distal end 624 of syringe 616 and screw drive 606 can be coupled to plunger 614. One or more operation buttons (not shown) on a side of device 600 can operate motor 608 in order to cause reverse or forward spin of screw drive 606 at one or more speeds. In some embodiments, buttons can be pressure sensitive and can allow for the reverse or forward spin of the screw at variable speeds dependent upon the pressure of a user's push. This can create negative pressure or positive pressure within syringe 616. In some embodiments, negative pressure can reach a maximum of about 635 mmHg, 25 inHg or 12.3 PSI, where 1 mmHg=1 Torr. An additional, separate button (not shown) can be used to control pulsatile or constant suction modes. It should be understood that buttons can be provided on the housing (not shown) or in another appropriate location.

Motor 608 can be modulated or otherwise controlled by controller module 610 that it is coupled with by a potentiometer (not shown). This can allow for customized manual control by an operator, or operation according to stored programs automatically, as initiated and terminated by a user. Controller module 610 can allow for pulsatile action of plunger 614 via backward and forward motion of screw drive 602 at different speeds. In some embodiments, these speeds can be faster than would generally be possible by manual manipulation without using the device. The pulsatile motion causing pressure changes in syringe 616 including vacuum pressure can loosen the desired, targeted fat cells from nearby tissues that they are coupled to, as has been proven with pump technology of other mediums and arts. In some embodiments, an electrical, mechanical or electromechanical shutoff can ensure that physical limitations of syringe 624 are not overreached. Once the desired, targeted quantity of fat cells are removed, the syringe is full or both, the user or operator can then transfer the removed fat cells in the body of the syringe to a different area or depression of the patient by moving the syringe and any coupled cannula to the new, desired location and pressing a forward injection button to operate the motor accordingly. In some embodiments, handheld micro-cannular suction devices may include pulsing operability while in others they may not.

In various embodiments, handheld micro-cannular suction devices can be custom sized for each differently sized syringe. Alternately, devices can be designed to accommodate a plurality of different sized syringes. In some embodiments, an ideally sized device housing can accommodate a syringe of about 10 cc that is able to achieve maximal negative pressure of about −559 mmHG, −22 inHg, or 10.8 PSI with a plunger pulled to 10-11 cc. After 10-12 cc of negative pressure, any further pull in larger syringes produces a slower increase in negative pressure and generally negative pressure reaches a plateau at about 30 cc of retraction, corresponding to −650 mmHg, -25.6 inHg or 12.6 PSI. Alternatively, although 12 cc syringes are less commonly found, a device can be sized to accommodate them.

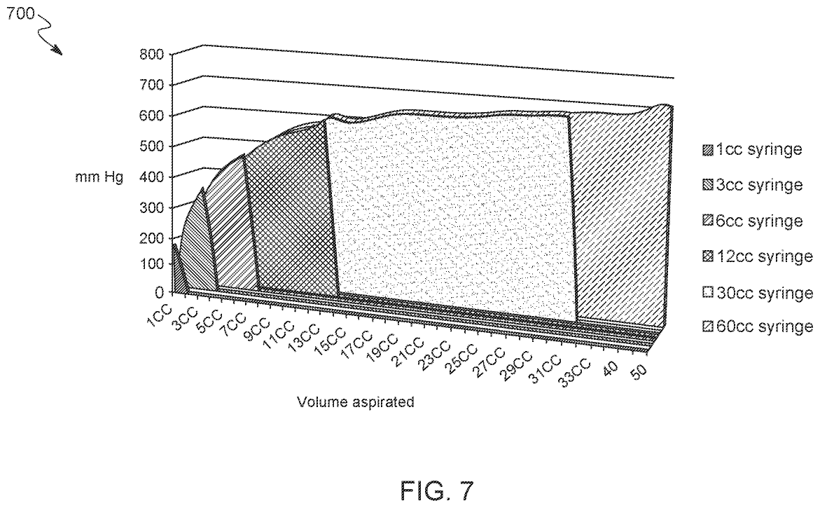

FIG. 7 shows an example embodiment of a volume aspirated versus mmHg graph 700. Graph 700 shows the plateauing of negative pressure for differently sized syringes. Graph 700 was displayed in “Quantification of Negative Pressures Generated by Syringes of Different Calibers Used for Liposuction”, Rodriguez RL, Conde-Green A, ASPS, Vol 130, #2, pp383e-384e. This paper, which is incorporated herein by reference in its entirety, describes the principle that each 1 cc of retraction on a syringe plunger creates the same amount of negative pressure regardless of whether a 1 cc, 3 cc, 10 cc, 20 cc or 60 cc syringe is used.

The concept of micro-liposuction has been theorized in the past but has essentially been defined as cannulas with outside diameters between 1-3 mm. In various embodiments described herein, microsuction cannulas can have an outside diameter less than 1.4 mm, which is equivalent to about a 17 gauge and which does not currently commonly exist. Infusion cannulas smaller in diameter than 19 gauge with only one hole do exist but are not used in suction procedures as fat cannot be removed through the small holes provided. As with other liposuction cannulas, the hole or holes at the tips of suction microcannulas can have one or an array of openings with a different number of holes, shapes, locations and sizes in various embodiments.

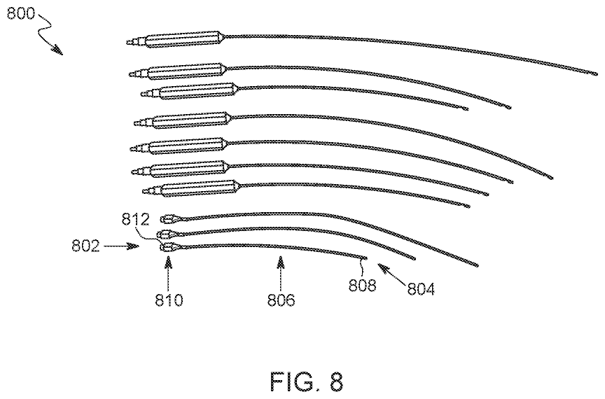

FIG. 8 shows an example embodiment diagram 800 of a plurality of curved cannulas with varying lengths and curvatures. As shown in the example embodiment, cannulas generally can have a cylindrical shape creating a hollow tube interior. This hollow tube is defined by an opening in a distal end 802, a cylindrical wall along a body section having a length and one or more curved areas having an arc length and curvature, and one or more holes or openings 808 near a distal end 804. A body section 810 can be shaped with edges or other grip related features and can have one or more coupling features 812 that are operable to removably engage with a syringe body, tubing, or other appropriate component. Coupling features 812 can be interior, exterior or both and can include threaded ridges, notches or others as appropriate to secure to the component to be coupled to.

Cannulas used in liposuction procedures can be metal, plastic or other appropriate materials or combinations of materials. In some embodiments, cannulas can provide ultrasonic capabilities, laser attributes or reciprocating movements. In some embodiments, a programmed microcontroller can cause one or more coupled step or brushless micro-motors that included or are coupled with screw drives or driveshafts to move back and forth or forward and backward in a manner, aligned and contiguous with the shaft of the syringe. This can therefore create pulsatile negative and positive pressures within a syringe.

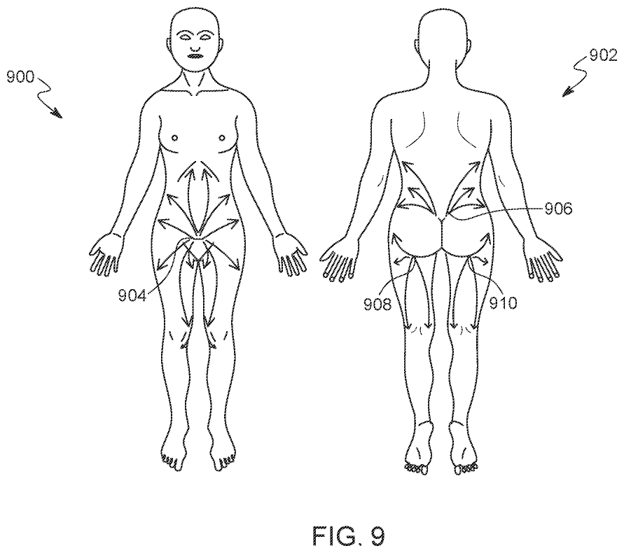

FIG. 9 shows an example embodiment of a human body frontal diagram 900 and human body backside diagram 902 showing various curved liposuction pathways for curved cannula. As described herein, the use of curved cannulas in liposuction procedures also allows a surgeon to reduce the number of incisions required in a procedure or use very few incisions, since the curved cannulas can spiral around certain bodily features. To elaborate, curved suction cannulas of different diameters and lengths can allow a surgeon to perform liposuction on areas around a patient's entire body from the chest to below the knees using a minimal number of incisions. As shown in the example embodiment, this can be as few as four incisions or less. In the frontal diagram 900, an incision 904 in the lower torso can allow for liposuction upward toward the chest, outward toward the hips and downward across the thighs and front of the legs on the outside and inside, even as far as below the inner knees. As shown in backside diagram, an incision 906 in the lower back can allow for liposuction upward across much of the back toward the shoulder blades. An incision 908 or 910 below the buttocks can allow for liposuction downward, inward and outward across much of the rear upper legs and upward around the buttocks.

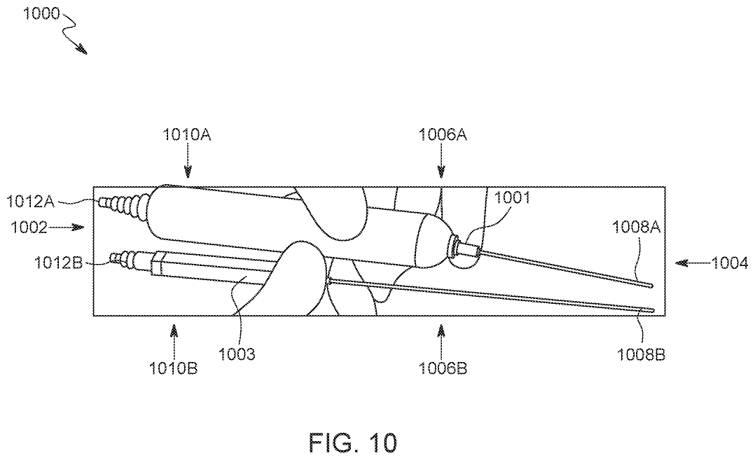

FIG. 10 shows an example embodiment diagram 1000 of two cannulas 1001, 1003. As shown in the example embodiment, each cannula 1001, 1003 can have a proximal end 1002 and distal end 1004. A gram liposuction cannula 1003 can be substantially similar to the cannulas described herein with respect to FIG. 8. A disposable cannula 1001 can be removably coupled with a reusable handle 1010a that has gripping features on its exterior surface.

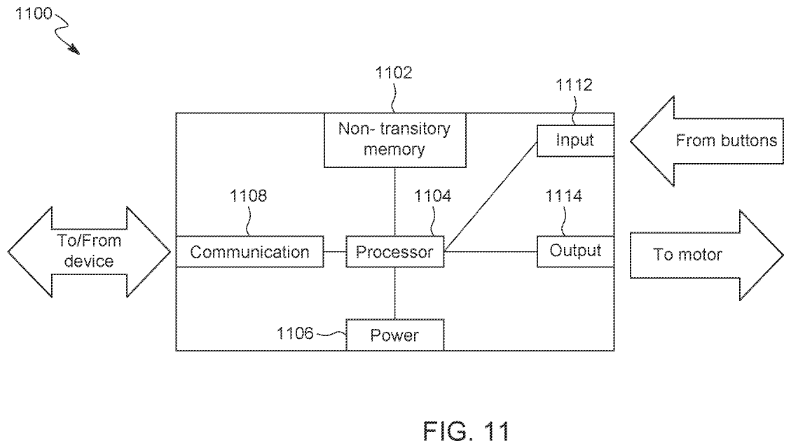

FIG. 11 shows an example embodiment of a controller architecture diagram 1100 for use with a handheld micro-cannular suction device, such as that described and shown in FIGS. 5-6. As shown in the example embodiment, non-transitory computer readable memory can store programs featuring instructions and also data regarding operations of the device. Non-transitory memory 1102 is coupled with a processor 1104 that can execute instructions stored in memory, process information, compute and perform other controller related functions. Processor receives power from power block 1106 that can be coupled with a battery in some embodiments. In some embodiments, it can be coupled with a wall plug or other fixed power source. Processor 1102 can receive orders or instructions from a user via input block 1112 in the form of button pushes or others, as appropriate. Processor 1104 can cause the controller to perform actions based on the instructions in the form of output block 1114, which can cause an electrically coupled motor to start, stop or otherwise change state based on the instructions received at input block 1112. In some embodiments, processor 1104 can be coupled with communication block 1108 that can communicate via a link with a computer or other device, such as a smart phone. This can be wired or wireless via computer networking protocols as known in the art or later developed such as Wi-Fi or Bluetooth. Communication block 1108 allows the handheld wireless micro-cannular suction device to be updated with new programs or instructions in some embodiments and for users to retrieve locally stored data regarding procedures from memory block 1102. Although not shown, it should be understood that other functional architectural blocks can be included in various embodiments, such as user interfaces including indicator lights, display screens, interactive touch screens and others that are coupled with processor block 1104 in an operative fashion to provide information to users.

It should be understood that controller architecture diagram 1100 is similar to one that could be provided for a controller for a peristaltic pulsator or piston driving generator, as described in FIGS. 1-4. As such, motors in the embodiments described with respect to those figures could be operated similarly via similar controllers when appropriately and operatively coupled via communication and electronic couplings. Additionally, it should be understood that operating buttons and other user interaction controls, though not specifically shown in the figures herein are contemplated and would be operatively and communicatively coupled to various components to provide the features and effects described herein.

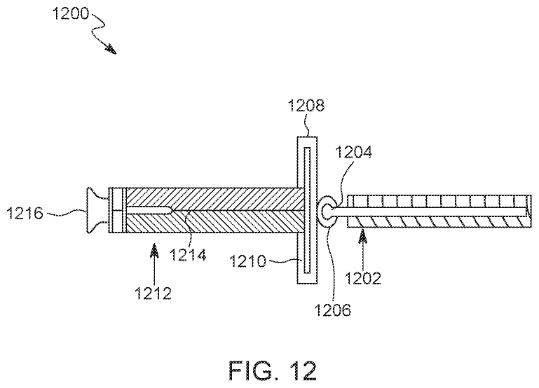

Another example embodiment of creating or augmenting pulsatile vacuum principles described herein is by using a new type of syringe with an incorporated side channel or hollow tube that is open at one end to the syringe's interior chamber near a plunger at a proximal end of the chamber and open near the distal tip of the syringe in the chamber. This will be described with respect to FIGS. 12-15.

FIG. 12 shows an example embodiment of a component diagram 1200 for coupling a syringe plunger 1212 with a screw drive 1202. As shown in the example embodiment, an adaptor clip 1208 can be removably coupled with a thumb rest 1210 of a plunger 1212 that is located at a proximal end of plunger 1212, opposite a distal end tip 1216. Adaptor clip 1208 can slide in a space in adaptor clip 1208 such that it is held securely. Clip 1208 can have straight edges that engage cross supports 1210 and thus prevent plunger 1212 from turning in adaptor clip 1208.

Opposite a plunger holding side of adaptor clip 1208 can be a removable or permanent tip holder 1206 for a tip 1204 of a screw drive 1202. As such, a distally located tip 1204 of screw drive 1202 can operate movement of adaptor clip 1208. In the example embodiment tip 1204 is a ball or sphere shape. As such, in the example embodiment the tip holder 1204 has a complementary and corresponding shape. The screw side of the clip would be attached to the screw via a rotating interface comprised of ball bearings or a simple spherical tip that rotates in a complementary housing of the clip.

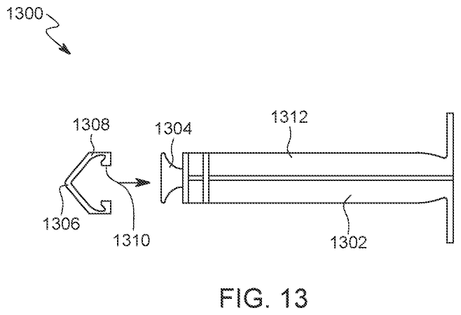

FIG. 13 shows an example embodiment diagram 1300 of a plunger 1302 and rubber tip 1306. In the example embodiment, plunger 1302 includes a plunger shaft tip 1304 at its distal end. Rubber tip 1306 can be shaped such that it points distally and can receive and couple to tip 1304 in a space on its proximal side. In some embodiments rubber tip can have a solid body 1308 that is bullet shaped, while in others body 1308 can be a series of arms separated from each other. One or more distally pointing protrusions 1310 can hold rubber tip substantially in place with tip 1304 when engaged such that any movement of rubber tip 1306 and plunger 1302 is done in unison.

Rotation of rubber tip 1306 can be mitigated, prevented or eliminated by allowing the plunger tip 1304 to rotate in rubber tip 1306. Coupling of rubber tip 1306 can be accomplished by clipping it onto plunger tip 1304 in such a way that any supporting internal syringe casing configurations needed to hold the rubber tip exterior walls to the syringe casing do not impede the rotation of the tip 1304 in the rubber tip 1306.

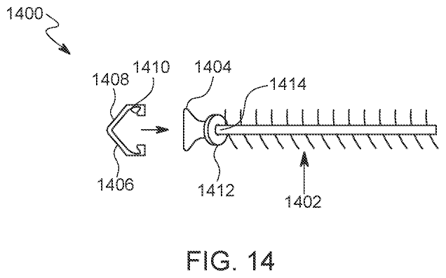

FIG. 14 shows an example embodiment diagram 1400 of a rubber tip 1406 and screw drive 1402. As shown in the example embodiment, rubber tip 1406 can be similar to one used with syringe plungers, as depicted and described with respect to FIG. 13. As such, 1306, 1308 and 1310 correspond similarly with numbers 1406, 1408 and 1410. In the example embodiment, rubber tip 1406 can be coupled with tip adaptor 1404. Tip adaptor 1404 allows rubber tip 1406 to be coupled with a screw drive 1402. At a proximal end of tip adaptor 1404 can be a removable or permanent tip holder 1412 for a tip 1414 of screw drive 1402. As such, a distally located tip 1414 of screw drive 1402 can operate movement of tip adaptor 1404 and rubber tip 1406. In the example embodiment, tip 1414 is a ball or sphere shape. As such, in the example embodiment the tip holder 1412 has a complementary and corresponding shape. As rubber tip 1406 is gripped firmly by a syringe casing's internal walls (not shown) in order to create a tight seal that prevents fluid escape, rubber tip 1406 is unlikely to or will not rotate as a coupled driveshaft (not shown) rotates. Mechanisms described in FIGS. 12-14 can eliminate rotation of a plunger rubber tip in various embodiments and can be used in unison in some embodiments.

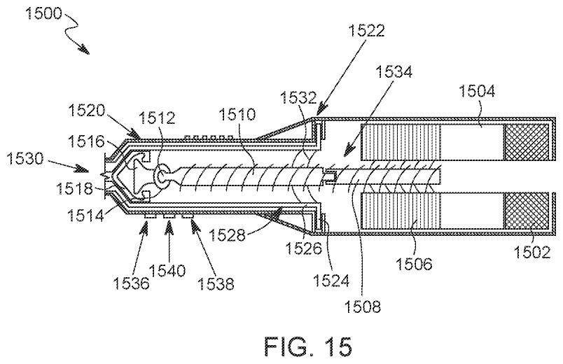

FIG. 15 shows an example embodiment diagram of a handheld micro-cannular suction device 1500. As shown in the example embodiment, device 1500 can include a battery 1502 that is coupled with and powers a controller module 1504, motor 1506 or both. Controller module 1504 can be a controller as described elsewhere herein with logic used to operate motor 1506.

Motor 1506 can include a first driveshaft 1508 the rotates in two directions when powered by motor 1506. A second driveshaft 1510 can be included in some embodiments that is coupled with first driveshaft 1508. As shown in the example embodiment, first driveshaft 1508 can be a device driveshaft, while second driveshaft 1510 can be a syringe pushing driveshaft. These can be coupled with a slot connection, D-ten clip or clip like securing mechanism.

A driveshaft tip 1512 can be coupled with a tip adaptor 1516 using a mechanism 1514 that receives and securely couples with driveshaft tip 1512. A rubber tip 1514 can be coupled with tip adaptor 1514 as described with respect to FIG. 14.

In operation, driveshaft tip 1512 can be propelled forward and backward within a hollow central cylinder 1534. Threading features within a central cylindrical chamber inside motor 1506 that is rotatably coupled with threading features can cause threading on complementary surfaces of first driveshaft 1508 to impel it proximally and distally.

An external housing 1520 can house one or more of the components and modules mentioned above with respect to FIG. 15. A syringe holding clip 1522 of syringe housing 1520 can hold one or more flanges 1524 or finger wings of a casing 1526 that are located opposite a distal end 1530 of a syringe 1528 where fluid or material is drawn in or pushed out. Here, custom or general purpose syringes 1528 can be inserted into clips 1524 via a door or opening (not shown) in a side of external housing 1520. When inserted from the side, the syringe holding clips 1522 accept the finger phalanges 1524 of the syringe. In general, flanges 1524 or finger wings of a casing 1526 of syringe 1528 can be inserted into a non-moving or stationary end of adapted handheld mechanical device 1500 such as those shown in FIGS. 5-6. It should be understood that external housings can be plastic, metal, combinations or other materials as appropriate.

Custom syringes 1528 can be manufactured with a screw plunger that is stabilized within the syringe casing with an internal screw stabilizer mechanism 1532. The driveshaft 1510 would thus be stabilized. When syringe 1528 is inserted into housing 1520, the slatted tip of the syringe screw 1510 would insert into a corresponding slit in the end of a screw driveshaft 1508 that is designed so that the threads line up perfectly. Some embodiments of these manufactured custom syringes can be designed to have standardized ergonomic casing diameters with standardized phalanges. They can also include different syringe casing lengths to adjust for different amounts of material to be aspirated, injected or both. Different external housing 1500 dimension sizes can also be manufactured to accommodate different sized syringes.

Alternatively, a side channel or hollow tube can be open at one end of a syringe casing's interior chamber near a plunger at a proximal end of the chamber and open to a location at or near a hub of an attachable cannula or needle. These types of features do not require major changes or modifications to standard syringes that are currently in use. Another end can have an airtight piston and plunger coupled to and driven by a powered motor. The piston and plunger can move forward and backward to increase and decrease negative or positive pressure in the syringe chamber according to the motor and any coupled gears. The piston and plunger action can also be driven by a round gear or eccentric gear that is operably coupled with a screw drive (as in FIGS. 5 and 6) that creates vacuum pressure. The piston and plunger action and forward and backward pulsations caused by the motor and screw drive can be timed to augment each other.

Also shown in FIG. 15 are forward button 1536, reverse button 1538 and pulsatile engaging button 1540. In some embodiments, when pulsatile engaging button or other buttons are selected, a light can be turned on or off to indicate the condition. Additionally, shown is a potentiometer 1542, operable as described elsewhere herein.

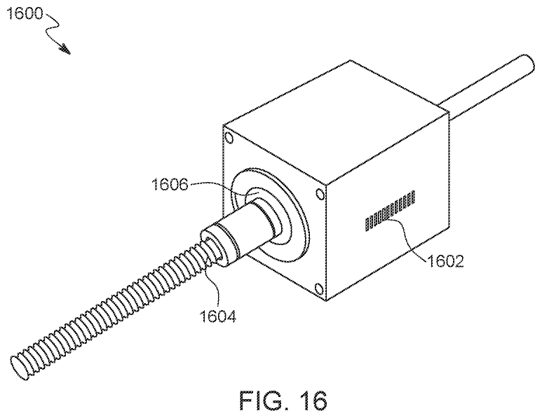

FIG. 16 shows an example embodiment of a drive motor 1600. As shown in the example embodiment, a motor body 1602 can include internal gears operable, when powered, to rotate a central section 1606 such that a driveshaft 1604 within rotates and moves along complementary threaded ridges.

It should be understood that couplings and seals as described herein can be removable or permanent as appropriate in various embodiments. Further, they can generally be accomplished through various means in various embodiments including mechanical couplings, adhesive couplings and various others.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances, entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

What is claimed is:

1. A portable, handheld liposuction system, comprising:
a motor, coupled to a controller and each of the motor and controller operably coupled to power source;
a driveshaft, coupled to the motor and operable to be rotated by the motor; and
a housing, operable to contain at least one of the motor, driveshaft and power source within a compartment; and
at least one user interface control on the housing;
wherein the controller comprises a processor, operable to execute instructions stored in non-transitory memory of the controller that cause the motor to turn the drive shaft in at least one direction based on user interaction with the user interface control, and
wherein the driveshaft is operable to cause at least one of expulsion of a fluid from a syringe body in a distal direction or aspiration of a fluid into a syringe body in a proximal direction.

2. The portable, handheld liposuction system of claim 1 wherein the driveshaft is operable to cause both of expulsion of a fluid from a syringe body in a distal direction and aspiration of a fluid into a syringe body in a proximal direction.

3. The portable, handheld liposuction system of claim 1, wherein the syringe body is at least partially held within the housing.

4. The portable, handheld liposuction system of claim 1, wherein the power source is a battery.

5. The portable, handheld liposuction system of claim 1, wherein the driveshaft does not move distally or proximally with respect to the motor when rotated by the motor.

6. The portable, handheld liposuction system of claim 5, further comprising:
a movable platform, coupled to the driveshaft, that moves distally or proximally with respect to the driveshaft when the driveshaft is rotated by the motor.

7. The portable, handheld liposuction system of claim 6, wherein the movable platform is coupled with a plunger of the syringe.

8. The portable, handheld liposuction system of claim 1, wherein the driveshaft is coupled with a plunger of the syringe.

9. The portable, handheld liposuction system of claim 1, wherein the driveshaft moves distally or proximally with respect to the motor when rotated by the motor.

10. The portable handheld liposuction system of claim 1, wherein the driveshaft is coupled with a rubber tip.

11. A portable, handheld liposuction device, comprising:
a motor, coupled to a controller and each of the motor and controller operably coupled to power source;
at least one driveshaft, coupled to the motor and operable to be rotated by the motor; and
a housing, operable to contain at least one of the motor, driveshaft and power source within a compartment; and
at least one user interface control on the housing for operating the motor and controller,
wherein the controller comprises a processor, operable to execute instructions stored in non-transitory memory of the controller that cause the motor to turn the drive shaft in at least one direction based on user interaction with the user interface control, and
wherein the driveshaft is operable to cause at least one of expulsion of a fluid from a syringe body in a distal direction or aspiration of a fluid into a syringe body in a proximal direction.

12. The portable, handheld liposuction device of claim 11, wherein the driveshaft is operable to cause both of expulsion of a fluid from a syringe body in a distal direction and aspiration of a fluid into a syringe body in a proximal direction.

13. The portable, handheld liposuction device of claim 11, wherein the syringe body is at least partially held within the housing.

14. The portable, handheld liposuction device of claim 11, wherein the power source is a battery.

15. The portable, handheld liposuction device of claim 11, wherein the driveshaft does not move distally or proximally with respect to the motor when rotated by the motor.

16. The portable, handheld liposuction device of claim 15, further comprising:
a movable platform, coupled to the driveshaft, that moves distally or proximally with respect to the driveshaft when the driveshaft is rotated by the motor.

17. The portable, handheld liposuction device of claim 16, wherein the movable platform is coupled with a plunger of the syringe.

18. The portable, handheld liposuction device of claim 11, wherein the driveshaft is coupled with a plunger of the syringe.

19. The portable, handheld liposuction device of claim 11, wherein the driveshaft moves distally or proximally with respect to the motor when rotated by the motor.

20. The portable, handheld liposuction device of claim 11, wherein the driveshaft is coupled with a rubber tip.
